(19) United States
(12) Patent Application Publication
(10) Pub. No.: US 2006/0121541 A1
(43) Pub. Date: Jun. 8, 2006

(54) Use of endosialin binding proteins to isolate endosialin positive cells

(76) Inventors: Luigi Grasso, Bala Cynwyd, PA (US); Gaurav Deshmukh, Norristown, PA (US); Nicholas C. Nicolaides, Boothwyn, PA (US); Philip M. Sass, Audubon, PA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/292,634

(22) Filed: Dec. 2, 2005

Related U.S. Application Data
(60) Provisional application No. 60/633,060, filed on Dec. 3, 2004.

(51) Int. Cl. G01N 33/574; C12N 5/06; C12N 5/08

(52) U.S. Cl. 435/7.23; 435/368; 435/338

(57) ABSTRACT

Monoclonal antibodies and other proteins that specifically bind to cells positive for endosialin are provided. The antibodies and proteins are useful in the isolation of endosialin-positive cells, particularly cells associated with neovascularization associated with cancer and neovascular disease. The invention is also related to cells that are isolated using monoclonal antibodies; antibody derivatives, such as chimeric and humanized monoclonal antibodies; antibody fragments; mammalian cells expressing the monoclonal antibodies, derivatives and fragments; and methods of detecting and isolating endosialin-positive cells using the antibodies, derivatives and fragments.

### CROSS-REFERENCE TO RELATED APPLICATIONS

This claims benefit of U.S. Provisional Application Ser. No. 60/633,060, filed Dec. 3, 2004, which is incorporated herein by reference in its entirety.

### FIELD OF THE INVENTION

This invention relates to the use of monoclonal antibodies and other proteins that specifically bind endosialin. The endosialin-binding proteins are useful in the isolation of endosialin-positive cells, particularly cells associated with neovascularization, for example, in cancer and neovascular disease. The invention is also related to cells that are isolated using monoclonal antibodies; antibody derivatives, such as chimeric and humanized monoclonal antibodies; antibody fragments; mammalian cells expressing the monoclonal antibodies, derivatives and fragments; and methods of detecting and isolating endosialin-positive cells using the antibodies, derivatives and fragments.

### BACKGROUND OF THE INVENTION

Angiogenesis is a regulated process involving the formation of new blood vessels. It plays an essential role in normal growth, embryonic development, wound healing, and other physiological processes (Yancopoulos et al. (2000) Nature 407(6801):242-8). Moreover, de novo angiogenesis is involved in several disease states including cancer, in which new “embryonic-like” blood vessels (the formation of which is referred to as neovascularization herein) appear that differ from normal vasculature with regards to structure and function (Hanahan and Weinberg (2000) Cell 100(1):57-70). A number of in vivo and in vitro studies have demonstrated biological differences between normal and disease-associated vasculature using various model systems of angiogenesis. Such differences offer the ability to develop novel anti-angiogenic compounds that can selectively inhibit vessel formation by embryonic-type, tumor-associated endothelial cells for therapy of cancer and neovascular disease.

In light of these opportunities for therapy, an intense search for potential targets that can specifically inhibit tumor and other neovascular disease-associated endothelial cell growth and function is ongoing. In an attempt to identify such targets, strategies have been designed to identify cell surface antigens of tumor stroma as well as to isolate specific proteins or RNA that are expressed in neovascular endothelial cells (Rettig et al. (1992) Proc. Natl. Acad. Sci. USA 89(22):10832-6; St. Croix et al. (2000) Science 289: 1197-1202). In 1992, Rettig et al. described monoclonal antibodies that recognize antigens on vessels within various cancer types (Rettig et al. (1992) Proc. Natl. Acad. Sci. USA 89(22):10832-6). One of these was designated FB5, which recognizes a ˜100 kDa protein on the surface of a neuroblastoma cell line, LA1-5s. FB5 is a murine IgG1 antibody that binds to endosialin (TEM1) and has been shown to recognize endothelial cells associated with a variety of different cancer types. Structural evaluation classified endosialin as a C-type lectin-like protein, composed of five globular extracellular domains (including a C-type lectin domain, one domain with similarity to the Sushi/ccp/scr pattern, and three EGF repeats). The protein also contains a mucin-like region, a transmembrane segment, and a short cytoplasmic tail. The protein appears to be a glycoprotein. Carbohydrate analysis shows that the endosialin core protein has an abundance of sialylated, O-linked oligosaccharides with similarities to sialomucin-like molecules.

Immunohistochemistry studies of malignant tissues have identified strong expression of endosialin in a number of neovascular endothelial cells in malignant tissues while expression in cell lines derived from embryonic-like endothelial cultures such as but not limited to HUVEC (Human Umbilical Vein Endothelial Cells) or HMVEC (Neonatal Dermal Microvascular Endothelial Cells) (Cambrex Corp.) is limited. Analysis of antibodies that can bind to endosialin have identified a subset of cells that express this antigen in endothelial cell cultures as well as a subset of antigen-expressing cells in normal tissue of patients. While these cells appear to be fibroblastic-like in morphology, their true identity is unknown. Subsequent work combined the complementarity determining regions (CDR) of the mouse FB5 onto a human IgG1 backbone to create a humanized antibody that is able to also bind to vessels within malignant tissues and a subset of cells in HMVEC cultures.

U.S. Pat. No. 5,342,757 also describes the FB5 antibody.

Neovascularization is associated with a number of disease states. In cancer it is believed that neovascularization is important to supply tumors with blood. In non-oncology diseases such as retinopathy and macular degeneration, uncontrolled neovascularization causes loss of sight (Wilkinson-Berka (2004) Curr. Pharm. Des. 10(27):3331-48; Das and McGuire (2003) Prog. Retin. Eye Res. 22(6):721-48). Moreover, several reports have identified a role for neovascularization in inflammatory disease (Paleolog and Miotla (1998) Angiogenesis 2(4):295-307). Methods to better define the embryonic-like endothelial and precursor cells associated with these disease states will lead to the development of novel drugs to treat these diseases. Conversely, neovascularization is associated with wound healing (Galiano et al. (2004) Am. J. Pathol. 164(6): 1935-47). Identification of precursor cells that can lead to new vessels for wound healing can offer the ability to identify drugs and factors that promote this process for enhancing wound treatment associated with trauma, burns and infection.

A difficult problem in effective antiangiogenic and proangiogenic therapy is the nondefined nature and biological processes of precursor cells associated with neovascularization (Bagley et al. (2003) Cancer Res. 63(18):5866-73). A mechanism for isolating and studying these cells would have utility in evaluating the effects of drugs that can specifically suppress or stimulate the normal biology of these cell types in contrast to adult-like endothelial cells associated with vessels in normal adult tissue (Asahara and Kawamoto (2004) Am. J. Physiol. Cell Physiol. 287(3):C572-9). A means to isolate and study these cells also would offer the ability to identify new and more specific therapeutic targets for treating neovascular disease and/or promoting neovascularization in wound healing. Primary tissues, tumors and subsets of endothelial cultures such as, but not limited to, HMVEC and HUVEC contain specialized cells that are able to be bound and isolated using anti-endosialin antibodies. A mechanism for the isolation and study of these cells is needed to better understand the biology of embryonic-like endothelial cells and precursors. Isolation of endosialin-positive cells also will enable their use in the study of anti-angiogenic and pro-angiogenic therapies. Moreover, these cells provide a reagent for analyzing activity of antibodies and binding proteins for therapy.

### SUMMARY OF THE INVENTION

It has been discovered that endosialin is expressed in a subset of cells located within normal tissues, tumor tissues, and a subset of primary endothelial cultures. Without wishing to be bound by any particular theory, it is believed that the formation of vessels associated with neovascular diseases such as cancer as well as angiogenesis associated with wound healing originate from these specialized cells. Studies have recognized that endosialin is localized to endothelia of tumors and in non-descript fibroblastic like cells (Rettig et al. (1992) Proc. Natl. Acad. Sci. USA 89(22):10832-6). The use of endosialin antibodies to isolate endosialin-positive cells for studying angiogenesis has not been described previously.

The invention provides the use of endosialin antibodies and endosialin binding proteins that specifically bind endosialin-positive cells for the isolation of such cells. In some embodiments are provided methods of isolating endosialin-positive cells by contacting a population of cells with an antibody that specifically binds endosialin. In some embodiments, the endosialin-positive cells bound to the endosialin antibody or binding protein are separated from unbound cells, for example, by removing the unbound cells from the population of cells or by removing the bound endosialin-positive cells from the population of cells. Separation of EPCs may be accomplished, for example, by FACS sorting, cell panning, column purification or removal of endosialin-negative cells. The endosialin-positive cells are, for example, isolated from primary endothelial cell populations, primary malignant tissues, normal tissues, primary culture cells, tissue culture cells, or immunosorted cells derived from a tissue. In some embodiments of the invention, the endosialin-positive cells contain recombinantly expressed endosialin. In some aspects, the methods of the invention further include analysis of an expression profile of the endosialin-positive cell directly after isolation; after culturing the cell; after culturing the cell in a stimulated state with growth factors and/or defined nutrients; or after contacting the cell with a test compound. The expression profile may be, for example, a RNA, cDNA, or protein profile.

The endosialin antibody or binding protein of the invention has an affinity of at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M.

The endosialin antibody or binding protein for use in the methods of the invention may be a human or non-human antibody. The endosialin antibody or binding protein may be a chimera, including but not limited, to a human-rodent (e.g., a human-mouse) chimeric antibody. The endosialin antibody of the invention may be a humanized antibody. The endosialin antibody or binding protein preferably includes at least one complementarity determining region that binds endosialin. The endosialin antibody or binding protein preferably includes a full-length heavy and light chain or a fragment having a Fab domain.

The invention also provides: hybridoma cells that express the endosialin antibodies or binding proteins of the invention; polynucleotides that encode the endosialin antibodies or binding proteins of the invention; vectors comprising the polynucleotides that encode the antibodies or binding proteins of the invention; and expression cells comprising the vectors of the invention. The antibody of the invention may arise from synthesis, recombinant phage, phage display and phage Fab libraries. The protein of the invention may arise from synthesis, recombinant phage or cDNA libraries.

Also provided by the invention are endosialin-positive cells isolated according to the methods of the invention. The endosialin-positive cells of the invention may be isolated from normal tissue, malignant tissue, inflamed tissue, diseased ocular tissue, primary cell cultures, or tissue culture cells. The endosialin-positive cells may be mammalian (e.g., rodent, human), plant, yeast, bacterial, fungal, or insect cells. The endosialin-positive cells may recombinantly express endosialin.

The invention also provides methods of producing an antibody or binding protein that specifically binds to the membrane bound form of endosialin whereby antibodies and/or binding proteins are screened for their ability to bind to cells expressing endosialin or cell membranes containing endosialin. The endosialin-expressing cells of the invention may be bacterial, yeast, plant, insect cells, and animal cells, preferably, mammalian cells.

Also provided by the invention are methods of identifying compounds that modulate the growth, differentiation, and/or biological activity of an endosialin-positive cell of the invention. The methods comprise contacting an endosialin-positive cell with a test compound and determining whether the compound modulates (i.e., stimulates or inhibits) the growth, differentiation, or biological activity of the cell. The test compound may be, for example, an antibody (e.g, monoclonal or polyclonal), an antibody fragment, an antibody having 1, 2, 3, 4, 5, or 6 complementarity determining regions that bind endosialin, a protein, a chemical, an antisense oligonucleotide, or a small interfering RNA molecule.

The invention further provides methods of using the endosialin-isolated cells in vitro comprising isolating endosialin-positive cells and studying their gene and protein expression profiles under unstimulated and stimulated growth/differentiation conditions, wherein said cells are (a) isolated from tissue or heterogeneous cultures and analyzed for gene or protein expression; (b) isolated grown in the presence of growth factors in liquid or semisolid culture and analyzed for gene or protein expression; (c) isolated grown in liquid or semisolid culture in the presence of pharmacologic agents to identify agents that can inhibit or promote endothelial cell activation; and (d) used in harvesting and isolating RNA and protein to study expression patterns in steady-state or induced culture conditions. The method may be used for studying a variety of biological activities of isolated cells for angiogenesis associated with various conditions, such as, but not limited to cancer. In preferred embodiments, the cells are isolated from mammalian tissues or endothelial cultures.

### BRIEF DESCRIPTION OF THE DRAWINGS

FIGS. 1A and 1B show immunohistochemical analysis of endosialin-positive cells of malignant tissue. Tumors were isolated from patients with colorectal cancer and flash frozen in liquid nitrogen. Samples were thin-sectioned and stained with anti-endosialin or isotype control antibody. As shown, vessels in the tumor (FIG. 1A) stained positive for endosialin while isotype control antibody stained serial section was negative (FIG. 1B).

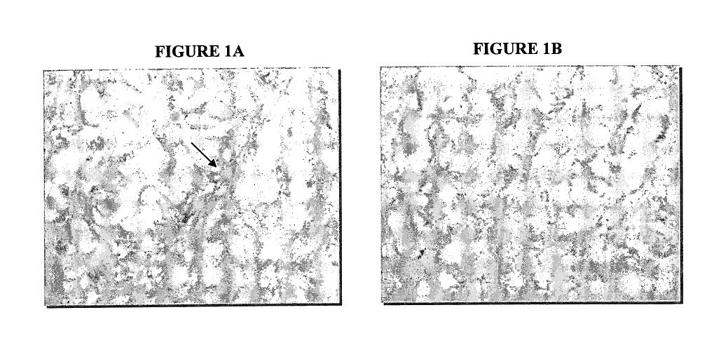

FIGS. 2A and 2B show immunohistochemical analysis of endosialin-positive cells of normal tissue. Normal tissues were isolated from patients by biopsy and flash frozen in liquid nitrogen. Samples were thin-sectioned and stained with anti-endosialin or isotype control antibody. As shown, normal tissue contained few EPCs (arrow, FIG. 2A) while isotype control antibody stained serial section were negative (FIG. 2B). Many normal tissues tested had a few EPCs as determined by in situ or antibody staining.

FIG. 3 shows isolation of endosialin-positive cells from primary endothelial cultures. HMVEC cultures were enriched for EPCs by panning. Endosialin-panned cultures were then compared to HMVEC parental cultures for percentage of endosialin-expressing cells. As shown, the panned culture had a much higher number of endosialin-positive cells as compared to the non-panned parental culture as determined by immunostaining via anti-endosialin antibody followed by a fluorescent conjugated secondary antibody. Cell number of each field was determined by light microscopy.

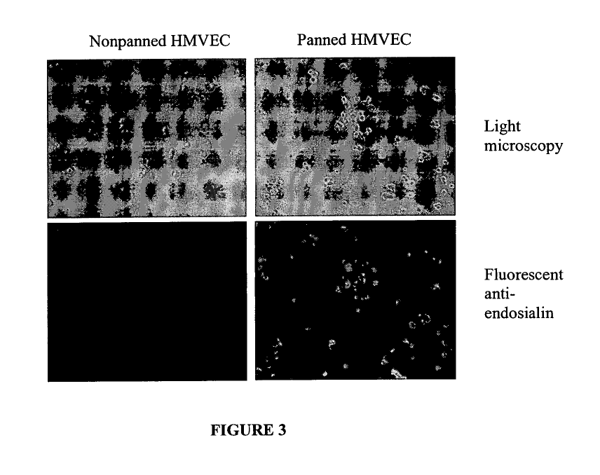

### DETAILED DESCRIPTION OF ILLUSTRATIVE EMBODIMENTS

The reference works, patents, patent applications, and scientific literature, including accession numbers to GenBank database sequences that are referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Standard reference works setting forth the general principles of recombinant DNA technology known to those of skill in the art include Ausubel et al., CURRENT PROTOCOLS IN MOLECULAR BIOLOGY, John Wiley & Sons, New York (1998); Sambrook et al., MOLECULAR CLONING: A LABORATORY MANUAL, 2D ED., Cold Spring Harbor Laboratory Press, Plainview, N.Y. (1989); Kaufman et al., Eds., HANDBOOK OF MOLECULAR AND CELLULAR METHODS IN BIOLOGY AND MEDICINE, CRC Press, Boca Raton (1995); McPherson, Ed., DIRECTED MUTAGENESIS: A PRACTICAL APPROACH, IRL Press, Oxford (1991).

Without wishing to be bound by any particular theory of operation, it is believed that endosialin-positive cells (EPCs) are precursors for vessels associated with neovascularization and neovascular disease such as cancer. Therefore, EPCs are specific to vessels associated with neovascular disease and potentially vessels involved in wound healing. Thus, EPCs serve as ideal cells to target for treatment of neovascular disease and cancer. Conversely, EPCs serve as ideal cells to target for promoting wound healing. EPCs are also useful in screens for compounds that modulate (i.e, stimulate or inhibit) the growth, differentiation, and/or biological activity of the cells.

Definitions

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

As used herein, the term “pharmacologic agent” refers to any agent that can be used to inhibit or stimulate that activity of EPCs including but not limited to small chemical entities, small interfering RNA (RNAi), antisense oligonucleotides, agonists, natural ligands, antibodies and endosialin binding proteins, vaccines and EPCs as whole cell vaccine.

As used herein, the term “endosialin-positive cell” (“EPC”) refers to a cell having endosialin present at or on its cell surface. Such a cell may be isolated from primary tissues or endothelial cultures using antibodies or binding proteins that can bind endosialin.

As used herein, the term “inhibition of growth, differentiation, and/or biological activity of EPCs” means a decrease in the total number of cells, the rate or extent of differentiation of cells, or the biological activity of cells, in culture, by about 5%, preferably about 10%, more preferably about 20%, more preferably about 30%, more preferably about 40%, more preferably about 50%, more preferably about 60%, more preferably about 70%, more preferably about 80%, more preferably about 90%, and most preferably about 100%. In vitro inhibition of cell growth may be measured by assays known in the art, such as the GEO cell soft agar assay.

As used herein, the term “stimulation of growth, differentiation, and/or biological activity of EPCs” means an increase in the total number of cells, the rate or extent of differentiation of cells, or the biological activity of cells, in culture, by about 5%, preferably about 10%, more preferably about 20%, more preferably about 30%, more preferably about 40%, more preferably about 50%, more preferably about 60%, more preferably about 70%, more preferably about 80%, more preferably about 90%, and most preferably about 100%. In vitro stimulation of cell growth may be measured by assays known in the art, such as the GEO cell soft agar assay.

The term “analysis” refers to analyzing RNA, cDNA, or protein profiles from EPCs. Analysis can be performed using any method used by those skilled in the art such as but not limited to differential expression methods such as microarray, cDNA libraries, SAGE, subtraction, or protein arrays.

The term “biological effect” refers to the inhibition or stimulation of a condition. A biological effect relieves to some extent one or more of the symptoms of the abnormal condition. In reference to the treatment of abnormal conditions, a biological effect can refer to one or more of the following: (a) an increase or decrease in the proliferation, growth, and/or differentiation of cells; (b) inhibition (i.e., slowing or stopping) of growth of tumor cells in vivo; (c) promotion of cell death; (d) inhibition of degeneration; (e) relieving to some extent one or more of the symptoms associated with the abnormal condition; and (f) enhancing the function of a population of cells. Pharmacologic agents and derivatives thereof described herein effectuate the biological effect alone or in combination with conjugates or additional components of the compositions of the invention.

As used herein, the term “about” refers to an approximation of a stated value within an acceptable range. Preferably the range is +/−5% of the stated value.

As used herein, the term “isolate” or derivatives thereof in reference, for example, to a cell, means enrichment (e.g., by panning) or purification of a specific cell-type within a heterogeneous population of cells.

As used herein, the term “contact” or “contacting” means bringing together, either directly or indirectly, a cell or compound into physical proximity to a cell or molecule of interest. Contacting may occur, for example, in any number of buffers, salts, solutions, or in a cell or cell extract.

As used herein, the term “normal tissue” refers to non-diseased tissue from mammalian embryos, fetal or adult sources.

As used herein, the term “diseased tissue” refers to tissue from mammalian embryos, fetal or adult sources that are infected, inflamed, or dysplastic. Tissues can be from but not limited to malignant sources; disease of the eye; or disease from tissues with infection and/or inflammation.

As used herein, the term “endothelial primary cultures” refers to cultures of cells derived from mammalian systems that have the ability to form vessels. These cells can be isolated from primary tissues, blood or cultures such as but not limited to HMVEC or HUVEC.

As used herein, the term “endosialin ligand” refers to any protein or biochemical compound that can bind to cell surface endosialin.

As used herein, the term “analytical cells” refers to EPCs that can be used to identify antibodies or binding proteins that can recognize endosialin expression on the cell surface. These cells can be primary cells isolated from tissues, blood or cultures such as but not limited to HMVEC or HUVEC or cells transfected with vectors expressing recombinant mammalian endosialin.

Endosialin Antibodies and Binding Proteins

The antibodies and binding proteins of the invention specifically bind endosialin present at or on the cell surface. In some embodiments, the antibodies and proteins bind to cells within normal tissue. In other embodiments, the antibodies and proteins bind to cells in diseased tissue. In other embodiments, the antibodies and proteins bind to cells in endothelial primary cultures.

Preferred antibodies and proteins suitable for use in the methods of the invention, include, for example, fully human antibodies, human antibody homologs, humanized antibody homologs, chimeric antibody homologs, Fab, Fab′, F(ab′)2 and F(v) antibody fragments, single chain antibodies, and monomers or dimers of antibody heavy or light chains, endosialin ligands or mixtures thereof that can isolate endosialin-positive cells from cell populations such as tissue or culture. The endosialin antibodies or binding proteins preferably contain at least 1, 2, 3, 4, 5, or 6 complementarity determining regions that bind endosialin.

The antibodies of the invention may include intact immunoglobulins of any isotype including types IgA, IgG, IgE, IgD, IgM (as well as subtypes thereof). The light chains of the immunoglobulin may be kappa or lambda.

The antibodies of the invention include portions of intact antibodies that retain antigen-binding specificity, for example, Fab fragments, Fab′ fragments, F(ab′)2 fragments, F(v) fragments, heavy chain monomers or dimers, light chain monomers or dimers, dimers consisting of one heavy and one light chain, and the like. Thus, antigen binding fragments, as well as full-length dimeric or trimeric polypeptides derived from the above-described antibodies are themselves useful.

The antibodies or binding proteins of the invention may be used alone or as a conjugate. Conjugation of antibodies and binding proteins is well-known in the literature.

The antibodies of the invention include derivatives that are modified, e.g., by the covalent attachment of any type of molecule to the antibody such that covalent attachment does not prevent the antibody from binding to its epitope. Examples of suitable derivatives include, but are not limited to glycosylated antibodies and fragments, acetylated antibodies and fragments, pegylated antibodies and fragments, phosphorylated antibodies and fragments, and amidated antibodies and fragments. The antibodies and derivatives thereof of the invention may themselves be derivatized by known protecting/blocking groups, proteolytic cleavage, linkage to a cellular ligand or other proteins, and the like. Further, the endosialin antibodies and derivatives thereof may contain one or more non-classical amino acids.

The invention also encompasses polyclonal antibodies such as those derived from peripheral blood of mammalian cell hosts. Such cells may be fused with myeloma cells, for example, to form hybridoma cells producing antibodies that can bind to EPCs.

Without wishing to be bound by any particular theory of operation, it is believed that the antibodies of the invention are particularly useful to bind endosialin positive cells due to a binding of the antibody to an extracellular epitope. This leads to a decrease in the dissociation constant (K_{d}) of the antibody. This is an especially good feature for targeting cells for isolation from tissue or tissue culture.

Methods of Producing Antibodies to Endosialin for Isolating Endosialin Positive Cells

Immunizing Animals

The invention also provides methods of producing monoclonal antibodies that specifically bind to cells positive for endosialin. Endosialin may be purified from cells or from recombinant systems using a variety of well-known techniques for isolating and purifying proteins. For example, but not by way of limitation, endosialin may be isolated based on the apparent molecular weight of the protein by running the protein on an SDS-PAGE gel and blotting the proteins onto a membrane. Thereafter, the appropriate size band corresponding to the endosialin may be cut from the membrane and used as an immunogen in animals directly, or by first extracting or eluting the protein from the membrane. As an alternative example, the protein may be isolated by size-exclusion chromatography alone or in combination with other means of isolation and purification. Other means of purification are available in such standard reference texts as Zola, MONOCLONAL ANTIBODIES: PREPARATION AND USE OF MONOCLONAL ANTIBODIES AND ENGINEERED ANTIBODY DERIVATIVES (BASICS: FROM BACKGROUND TO BENCH), Springer-Verlag Ltd., New York, 2000; BASIC METHODS IN ANTIBODY PRODUCTION AND CHARACTERIZATION, Chapter 11, “Antibody Purification Methods,” Howard and Bethell, Eds., CRC Press, 2000; ANTIBODY ENGINEERING (SPRINGER LAB MANUAL), Kontermann and Dubel, Eds., Springer-Verlag, 2001.

One strategy for generating antibodies against endosialin involves immunizing animals with the endosialin. Animals so immunized will produce antibodies against the protein. Standard methods are known for creating monoclonal antibodies including, but are not limited to, the hybridoma technique (see Kohler & Milstein (1975) Nature 256:495-497); the trioma technique; the human B-cell hybridoma technique (see Kozbor et al. (1983) Immunol. Today 4:72) and the EBV hybridoma technique to produce human monoclonal antibodies (see Cole, et al. in MONOCLONAL ANTIBODIES AND CANCER THERAPY, Alan R. Liss, Inc., 1985, pp. 77-96).

Screening for Antibody or Binding Protein Specificity

Screening for antibodies or binding proteins that specifically bind to cells expressing endosialin may be accomplished using an enzyme-linked immunosorbent assay (ELISA) in which microtiter plates are coated with the endosialin. Antibodies or binding proteins from positively reacting clones can be further screened for reactivity in a cell-based ELISA assay to identify those that can bind cells positive for endosialin. Clones that produce antibodies or binding proteins that are reactive to EPCs are selected for further expansion and development.

Confirmation of reactivity of the antibodies or binding proteins to endosialin may be accomplished, for example, using an immunohistochemistry study in which cells from tissue or tissue culture are prepared and probed with the putative anti-endosialin antibodies or specific binding proteins. Reactivity with tissues or cells under non-reducing conditions confirms specificity of reactivity for endosialin.

The antibodies, derivatives and binding proteins thereof of the invention have binding affinities that include a dissociation constant (K_{d}) of less than 1×10⁻². In some embodiments, the K_{d} is less than 1×10⁻³. In other embodiments, the K_{d} is less than 1×10⁻⁴. In some embodiments, the K_{d} is less than 1×10⁻⁵. In still other embodiments, the K_{d} is less than 1×10⁻⁶. In other embodiments, the K_{d} is less than 1×10⁻⁷. In other embodiments, the K_{d} is less than 1×10⁻⁸. In other embodiments, the K_{d} is less than 1×10⁻⁹. In other embodiments, the K_{d} is less than 1×10⁻¹⁰. In still other embodiments, the K_{d} is less than 1×10⁻¹¹. In some embodiments, the K_{d} is less than 1×10⁻¹². In other embodiments, the K_{d} is less than 1×10⁻¹³. In other embodiments, the K_{d} is less than 1×10⁻¹⁴. In still other embodiments, the K_{d} is less than 1×10⁻¹⁵.

Production of Antibodies

Antibodies of the invention may be produced in vivo or in vitro. For in vivo antibody production, animals are generally immunized with an immunogenic portion of endosialin or a peptide from the endosialin. The antigen is generally combined with an adjuvant to promote immunogenicity. Adjuvants vary according to the species used for immunization. Examples of adjuvants include, but are not limited to: Freund's complete adjuvant (“FCA”), Freund's incomplete adjuvant (“FIA”), mineral gels (e.g., aluminum hydroxide), surface active substances (e.g., lysolecithin, pluronic polyols, polyanions), peptides, oil emulsions, keyhole limpet hemocyanin (“KLH”), dinitrophenol (“DNP”), and potentially useful human adjuvants such as Bacille Calmette-Guerin (“BCG”) and corynebacterium parvum. Such adjuvants are also well known in the art.

Immunization may be accomplished using well-known procedures. The dose and immunization regimen will depend on the species of mammal immunized, its immune status, body weight, and/or calculated surface area, etc. Typically, blood serum is sampled from the immunized mammals and assayed for endosialin antibodies using appropriate screening assays as described below, for example.

Splenocytes from immunized animals may be immortalized by fusing the splenocytes (containing the antibody-producing B cells) with an immortal cell line such as a myeloma line. Typically, the myeloma cell line is from the same species as the splenocyte donor. In one embodiment, the immortal cell line is sensitive to culture medium containing hypoxanthine, aminopterin and thymidine (“HAT medium”). In some embodiments, the myeloma cells are negative for Epstein-Barr virus (EBV) infection. In preferred embodiments, the myeloma cells are HAT-sensitive, EBV negative, and Ig expression negative. Any suitable myeloma may be used. Murine hybridomas may be generated using mouse myeloma cell lines (e.g., the P3-NS1/1-Ag4-1, P3-x63-Ag8.653 or Sp2/O-Ag14 myeloma lines). These murine myeloma lines are available from the ATCC. These myeloma cells are fused to the donor splenocytes using polyethylene glycol (“PEG”), preferably 1500 molecular weight polyethylene glycol (“PEG 1500”). Hybridoma cells resulting from the fusion are selected in HAT medium which kills unfused and unproductively fused myeloma cells. Unfused splenocytes die over a short period of time in culture. In some embodiments, the myeloma cells do not express immunoglobulin genes.

Hybridomas producing a desired antibody which are detected by screening assays such as those described below, may be used to produce antibodies in culture or in animals. For example, the hybridoma cells may be cultured in a nutrient medium under conditions and for a time sufficient to allow the hybridoma cells to secrete the monoclonal antibodies into the culture medium. These techniques and culture media are well known by those skilled in the art. Alternatively, the hybridoma cells may be injected into the peritoneum of an unimmunized animal. The cells proliferate in the peritoneal cavity and secrete the antibody, which accumulates as ascites fluid. The ascites fluid may be withdrawn from the peritoneal cavity with a syringe as a rich source of the monoclonal antibody.

Another non-limiting method for producing human antibodies is described in U.S. Pat. No. 5,789,650 which describes transgenic mammals that produce antibodies of another species (e.g., humans) with their own endogenous immunoglobulin genes being inactivated. The genes for the heterologous antibodies are encoded by human immunoglobulin genes. The transgenes containing the unrearranged immunoglobulin encoding regions are introduced into a non-human animal. The resulting transgenic animals are capable of functionally rearranging the transgenic immunoglobulin sequences and producing a repertoire of antibodies of various isotypes encoded by human immunoglobulin genes. The B-cells from the transgenic animals are subsequently immortalized by any of a variety of methods, including fusion with an immortalizing cell line (e.g., a myeloma cell).

Antibodies against endosialin may also be prepared in vitro using a variety of techniques known in the art. For example, but not by way of limitation, fully human monoclonal antibodies against endosialin may be prepared by using in vitro-primed human splenocytes (Boerner et al. (1991) J. Immunol. 147:86-95).

Alternatively, for example, the antibodies of the invention may be prepared by “repertoire cloning” (Persson et al. (1991) Proc. Nat. Acad. Sci. USA 88:2432-2436; and Huang and Stollar (1991) J. Immunol. Methods 141:227-236). Further, U.S. Pat. No. 5,798,230 describes preparation of human monoclonal antibodies from human B antibody-producing B cells that are immortalized by infection with an Epstein-Barr virus that expresses Epstein-Barr virus nuclear antigen 2 (EBNA2). EBNA2, required for immortalization, is then inactivated resulting in increased antibody titers.

In another embodiment, antibodies against endosialin are formed by in vitro immunization of peripheral blood mononuclear cells (“PBMCs”). This may be accomplished by any means known in the art, such as, for example, using methods described in the literature (Zafiropoulos et al. (1997) J. Immunological Methods 200:181-190).

In some embodiments of the invention, the procedure for in vitro immunization is supplemented with directed evolution of the hybridoma cells in which a dominant negative allele of a mismatch repair gene such as PMS1, PMS2, PMS2-134, PMSR2, PMSR3, MLH1, MLH2, MLH3, MLH4, MLH5, MLH6, PMSL9, MSH1, and MSH2 is introduced into the hybridoma cells after fusion of the splenocytes, or to the myeloma cells before fusion. Cells containing the dominant negative mutant will become hypermutable and accumulate mutations at a higher rate than untransfected control cells. A pool of the mutating cells may be screened for clones that produce higher affinity antibodies, or that produce higher titers of antibodies, or that simply grow faster or better under certain conditions. The technique for generating hypermutable cells using dominant negative alleles of mismatch repair genes is described in U.S. Pat. No. 6,146,894, issued Nov. 14, 2000. Alternatively, mismatch repair may be inhibited using the chemical inhibitors of mismatch repair described by Nicolaides et al. in WO 02/054856 “Chemical Inhibitors of Mismatch Repair” published Jul. 18, 2002. The technique for enhancing antibodies using the dominant negative alleles of mismatch repair genes or chemical inhibitors of mismatch repair may be applied to mammalian expression cells expressing cloned immunoglobulin genes as well. Cells expressing the dominant negative alleles can be “cured” in that the dominant negative allele can be inactivated, turned off if inducible, or eliminated from the cell and the like such that the cells become genetically stable once more and no longer accumulate mutations at the abnormally high rate.

Endosialin-Positive Cells

The endosialin-positive cells of the invention include cells that naturally express endosialin or cells transfected with a recombinant plasmid expressing endosialin. Primary cells of the invention can be isolated from tissues or purchased from vendors selling endothelial cells, such as but not limited to HUVEC or HMVEC. Transfected cells of the invention include any insect expression cell line known, such as for example, Spodoptera frugiperda cells. The expression cell lines may also be yeast cell lines, such as, for example, Saccharomyces cerevisiae and Schizosaccharomyces pombe cells. The expression cells may also be plant, bacterial, fungal, or mammalian cells such as, for example Chinese Hamster Ovary, baby hamster kidney cells, human embryonic kidney line 293, normal dog kidney cell lines, normal cat kidney cell lines, monkey kidney cells, African green monkey kidney cells, COS cells, and non-tumorigenic mouse myoblast G8 cells, fibroblast cell lines, myeloma cell lines, mouse NIH/3T3 cells, LMTK31 cells, mouse sertoli cells, human cervical carcinoma cells, buffalo rat liver cells, human lung cells, human liver cells, mouse mammary tumor cells, TRI cells, MRC 5 cells, and FS4 cells.

Methods of Isolating Endosialin-Positive Cells

The invention provides the use of endosialin antibodies and endosialin binding proteins that specifically bind to the endosialin-positive cells for the isolation of such cells. The methods may comprise, for example, contacting a population of cells with an antibody or protein that specifically binds endosialin. The bound endosialin-positive cells may be separated from the unbound cells, for example, by removing the unbound cells from the population of cells or by removing the bound endosialin-positive cells from the population of cells. Isolation of endosialin-positive cellsmay be accomplished, for example, by panning as described herein.

The endosialin-positive cells may be isolated, for example, from primary endothelial cell populations, primary malignant tissues, normal tissues, primary culture cells, tissue culture cells, or immunosorted cells derived from a tissue. The endosialin-positive cells may recombinantly express endosialin. The cell populations from which endosialin positive cells are isolated according to the methods of the invention may be mammalian, preferably human or rodent, insect, bacterial, fungal, plant, or yeast.

The methods of isolating endosialin-positive cells may further include analysis of an expression profile of the isolated endosialin-positive cell directly after isolation; after culturing the cell; after culturing the cell in a stimulated state with growth factors and/or defined nutrients; or after contacting the cell with a test compound. The expression profile may be an RNA, cDNA, or protein profile.

The endosialin antibodies or binding proteins used in the methods of the invention preferably have an affinity of at least about 1×10⁻⁷ M, at least about 1×10⁻⁸ M, at least about 1×10⁻⁹ M, at least about 1×10⁻¹⁰ M, at least about 1×10⁻¹¹ M, or at least about 1×10⁻¹² M. The endosialin antibodies or binding proteins for use in the methods of the invention may be human or non-human. The endosialin antibodies or binding proteins may be chimeric, including but not limited, to a human-rodent (e.g., a human-mouse) chimeric antibody. The endosialin antibodies for use in the invention may be humanized antibodies. The endosialin antibodies or binding proteins preferably include at least one complementarity determining region that binds endosialin. The endosialin antibodies or binding proteins preferably include a full-length heavy and light chain or a fragment having a Fab domain.

In some embodiments, the endosialin may comprise an amino acid sequence of SEQ ID NO: 2, 4, or 6. In some embodiments, the endosialin may be encoded by a nucleotide sequence comprising SEQ ID NO:1, 3, or 5. The methods of the invention may be used to isolate endosialin-positive cells to study biological activities of the cells in angiogenesis. “Biological activity” as used herein refers to the level of a particular function (for example, enzymatic activity) of a cell, molecule, or pathway of interest in a biological system. “Wild-type biological activity” refers to the normal level of function of a cell, molecule or pathway of interest. “Reduced biological activity” refers to a decreased level of function of a cell, molecule, or pathway of interest relative to a reference level of biological activity of that cell, molecule, or pathway. For example, reduced biological activity may refer to a decreased level of biological activity relative to the wild-type biological activity of a cell, molecule or pathway of interest. “Increased biological activity” refers to an increased level of function of a cell, molecule or pathway of interest relative to a reference level of biological activity of that cell, molecule or pathway. For example, increased biological activity may refer to an increased level of biological activity relative to the wild-type biological activity of a cell, molecule or pathway of interest. In some embodiments of the invention, the endosialin-positive cells are analyzed after isolation or after culturing isolated cells in vitro under a variety of growth conditions. The cells can also be used in methods for identifying compounds such as growth factors, pharmacologic agents, or combinations thereof, that can inhibit or stimulate the cells to promote or inhibit their growth, stasis, differentiation, and biological activity in angiogenesis. In some embodiments, the cells can be used to identify cell surface factors for diagnosis and treatment of neovascular disease.

The methods of the invention are suitable for use in humans and non-human animals identified as having EPCs. Cells may be living or fixed. Non-human animals which benefit from the invention include pets, exotic (e.g., zoo animals) and domestic livestock. Preferably the non-human animals are mammals such as but not limited to rodents (e.g., mice, rats).

The invention is suitable for use with a human or animal patient that is identified as having a dysplastic disorder that is marked by increased expression of endosialin in the diseased tissue in relation to normal tissues. Malignant tissues that may be used for isolation of EPCs include but are not limited to ovarian tumors, renal tumors, colorectal tumors, pancreatic tumors, prostate tumors, lung tumors, fallopian tube tumors, uterine tumors, and brain tumors. Tissues affected with inflammation may also be used such as but not limited to arthritic tissue or tissue from inflamed airways. Eye tissue having neovascularization may also be used. Conversely normal tissue may also be used for isolation.

The endosialin binding proteins and antibodies and derivatives thereof for use in the invention may be used to isolate endosialin-positive cells. The antibodies and derivatives of the invention may be used to isolate EPCs using various methods of cell separation including FACS sorting, cell panning, column purification or removal of endosialin-negative cells known by those skilled in the art.

Cells from whole tissue may be isolated from fresh biopsies or fixed slides (Florell et al. (2001) Mod Pathol. 14(2):116-28).

EPCs isolated according to the methods of the invention may be characterized by, for example, an expression profile. The analysis may occur directly after isolation; after culturing the cell; after culturing the cell in a stimulated state with growth factors and/or defined nutrients; or after contacting the cell with a test compound. The expression profile may be, for example, a RNA, cDNA, or protein profile. Gene and protein expression profiles of the EPCs may be analyzed, for example, under unstimulated and stimulated growth/differentiation conditions. For example, EPCs may be isolated grown in the presence and absence of growth factors in liquid or semisolid culture and analyzed for gene or protein expression or isolated grown in liquid or semisolid culture in the presence and absence of pharmacologic agents to identify agents that can inhibit or promote endothelial cell activation. EPCs also may be used in harvesting and isolating RNA and protein to study expression patterns in steady-state or induced culture conditions. The methods may be used for studying a variety of biological activities of isolated cells for angiogenesis associated with various conditions, such as, but not limited to cancer and neovascular disease.

Use of EPCs to Identify Endosialin-Specific Antibodies and Binding Proteins

The methods of the invention are suitable for use in identifying antibodies and binding proteins that can specifically bind to membrane-bound endosialin. For example, test antibodies or binding proteins are contacted with EPCs or membranes containing endosialin. Specificity of binding can be determined, for example, by a comparison with antibody or protein binding to cells or membranes not expressing or having present at their surface endosialin. Detection of binding can be by ELISA, immunohistochemistry, FACS as well as other methods used for protein detection by those skilled in the art. Cells may be living or fixed. Cells may be primary cells or transfected cells. Cells may be human or non-human.

Use of EPCs to Identify Compounds that Modulate EPCs

EPCs isolated according to the methods of the invention may be used in methods to identify compounds that modulate (i.e., stimulate or inhibit) the growth, differentiation, and/or biological activity of an endosialin-positive cell. For example, an endosialin-positive cell may be contacted with a test compound followed by evaluation of the growth, differentiation, or biological activity of the cell. Inhibition of growth, differentiation, and/or biological activity of EPCs may be determined by measuring a decrease in the total number of cells, the rate or extent of differentiation of cells, or the biological activity of cells in culture. Stimulation of growth, differentiation, and/or biological activity of EPCs may be determined by detecting an increase in the total number of cells, the rate or extent of differentiation of cells, or the biological activity of cells in culture. For example, in vitro inhibition or stimulation of cell growth may be measured by assays known in the art, such as the GEO cell soft agar assay.

The test compound may be, for example, an antibody (e.g, monoclonal or polyclonal), an antibody fragment, an antibody having 1, 2, 3, 4, 5, or 6 complementarity determining regions that bind endosialin, a protein, a chemical, an antisense oligonucleotide, or a small interfering RNA molecule.

### LIST OF REFERENCES

Conejo-Garcia J R, Buckanovich R J, Benencia F, Courreges M C, Rubin S C, Carroll R G, Coukos G. Vascular Leukocytes Contribute to Tumor Vascularization. Blood. 2004 Sep. 9.

Carson-Walter E B, Watkins D N, Nanda A, Vogelstein B, Kinzler K W, St Croix B. Cell surface tumor endothelial markers are conserved in mice and humans. Cancer Res. 2001 Sep. 15; 61(18):6649-55.

Opavsky R, Haviernik P, Jurkovicova D, Garin M T, Copeland N G, Gilbert D J, Jenkins N A, Bies J, Garfield S, Pastorekova S, Oue A, Wolff L. Molecular characterization of the mouse Tem1/endosialin gene regulated by cell density in vitro and expressed in normal tissues in vivo. J. Biol. Chem. 2001 Oct. 19; 276(42):38795-807.

Christian S, Ahorn H, Koehler A, Eisenhaber F, Rodi H P, Garin-Chesa P, Park J E, Rettig W J, Lenter M C. Molecular cloning and characterization of endosialin, a C-type lectin-like cell surface receptor of tumor endothelium. J. Biol. Chem. 2001 Mar. 9; 276(10):7408-14.

Rettig W J, Garin-Chesa P, Healey J H, Su S L, Jaffe E A, Old L J. Identification of endosialin, a cell surface glycoprotein of vascular endothelial cells in human cancer. Proc. Natl. Acad. Sci. USA 1992 Nov. 15; 89(22):10832-6.

St Croix B, Rago C, Velculescu V, Traverso G, Romans K E, Montgomery E, Lal A, Riggins G J, Lengauer C, Vogelstein B, Kinzler K W. Genes expressed in human tumor endothelium. Science. 2000 Aug. 18; 289(5482): 1121-2.

Watt S M, Chan J Y. CD164—a novel sialomucin on CD34+ cells. Leuk. Lymphoma. 2000 March; 37(1-2):1-25.

Kurosawa N, Kanemitsu Y, Matsui T, Shimada K, Ishihama H, Muramatsu T. Genomic analysis of a murine cell-surface sialomucin, MGC-24/CD164. Eur. J. Biochem. 1999 Oct. 1; 265(1):466-72.

Buhring H J, Seiffert M, Bock T A, Scheding S, Thiel A, Scheffold A, Kanz L, Brugger W. Expression of novel surface antigens on early hematopoietic cells. Ann. N.Y. Acad. Sci. 1999 Apr. 30; 872:25-38; discussion 38-9.

Zannettino A C, Buhring H J, Niutta S, Watt S M, Benton M A, Simmons P J. The sialomucin CD164 (MGC-24v) is an adhesive glycoprotein expressed by human hematopoietic progenitors and bone marrow stromal cells that serves as a potent negative regulator of hematopoiesis. Blood. 1998 Oct. 15; 92(8):2609-12.

Watt S M, Buhring H J, Rappold I, Chan J Y, Lee-Prudhoe J, Jones T, Zannettino AC, Simmons P J, Doyonnas R, Sheer D, Butler L H. CD164, a novel sialomucin on CD34(+) and erythroid subsets, is located on human chromosome 6q21. Blood. 1998 Aug. 1; 92(3):849-66.

The following Examples are provided to illustrate the present invention, and should not be construed as limiting thereof.

### EXAMPLES

### Example 1

Use of antibodies to detect EPCs was shown by immunohistochemistry of malignant tissues. The anti-endosialin or normal IgG antibody was applied to freshly frozen human colorectal cancer tissues at two concentrations (0.5 μg/mL and 2.5 μg/mL). Phosphate-buffered saline [PBS (0.15M NaCl, pH 7.2)]+1% bovine serum albumin served as the diluent for the primary antibodies. Tissues were embedded in TISSUE-TEK® O.C.T. medium, frozen on dry ice, and stored in sealed plastic bags below −70° C. Tissues were sectioned at approximately 5 μm, and fixed for 10 minutes in room temperature acetone. Slides were stored below −70° C. until staining. Just prior to staining, slides were fixed for 10 seconds in 10% neutral-buffered formalin.

Cryosections were rinsed twice in phosphate-buffered saline (PBS [0.15M NaCl, pH 7.2]). Endogenous peroxidase was blocked by incubating the slides with the peroxidase solution provided in the Dako EnVision Kit for 5 minutes and rinsing twice in PBS (0.15M NaCl, pH 7.2). Next, the slides were treated with a protein block designed to reduce nonspecific binding for 20 minutes. The protein block was prepared as follows: PBS (0.15M NaCl, pH 7.2); 0.5% casein; 1% bovine serum albumin (BSA); and 1.5% normal goat serum. Following the protein block, the primary antibody (test article, negative control antibody, or none [buffer alone as the assay control]) was applied at room temperature for one hour. Next, the slides were rinsed two times with PBS (0.15M NaCl, pH 7.2), treated with the peroxidase-labeled goat anti-IgG polymer supplied in the DAKO ENVISION Kit for 30 minutes (ENVISION polymer used at the concentration provided by manufacturer), rinsed two times with PBS (0.15M NaCl, pH 7.2), and treated with the substrate-chromogen (DAB) solution supplied in the DAKO ENVISION Kit for 8 minutes. All slides were rinsed in water, counterstained with hematoxylin, dehydrated and coverslipped for interpretation.

### Example 2

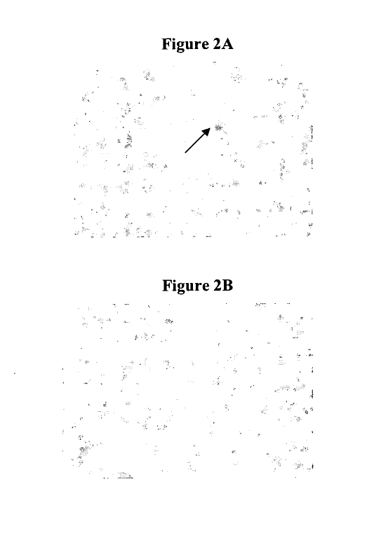

Use of antibodies to detect EPCs was shown by immunohistochemistry of normal tissues. Briefly, normal tissue specimens were sectioned by cryostat and analyzed for endosialin expression as described above. As shown in FIG. 2, normal tissues contained very few fibroblast/dendritic-like cells that are endosialin-positive albeit not as robustly or homogenously as was observed in the vessels within tumors. These cells are useful to study the effects of neovascularization and can be isolated for gene expression to study profiles of cell growth, differentiation, migration or signature identification. They can be studied in vivo, ex vivo or in vitro using methods known by those skilled in the art as well as those listed below.

### Example 3

To demonstrate that proteins that can bind to endosialin serve as an effective way for enriching endothelial or fibroblast-like endosialin expressing cells, Human MicroVascular Endothelial Cells (HMVECs) were panned using an antibody that can bind to endosialin to isolate an enriched population of EPCs from a starting pool containing 5-10% endosialin-positive cells. The method or specific reagents exemplified below are not intended to limit the present invention of the use of endosialin antibodies in isolating EPCs.

96-well plates were coated in sterile conditions with goat anti human IgG Fcγ. Next, 20 μg/ml of a human anti-endosialin antibody was added to the plates, three wells (A, B, C) without the antibody serving as controls, and incubated for 1 hour at 4° C. HMVECs were harvested from 10 cm petri dish cultures with DPBS/EDTA rather than trypsin to avoid any damage to the cell membranes, thereby leaving the endosialin cell surface proteins intact. Pooled cells were plated at two different concentrations, either 100,000 cells/well or 50,000 cells/well in the 96-well plates after aspirating and washing off any unbound anti-endosialin antibody. Cells were incubated in plates for one hour at 4° C. Plates were then washed with DPBS/FBS four times (until control wells A, B, and C showed no cells within the wells). The 50,000 cells/well plate showed very few cells, while the 100,000 cells/well wells contained a number of cells attached to plate. Cells were then incubated for three days in appropriate growth media. The control B and C wells were picked from the 100,000 cells/well plates for immunostaining. Calcein, AM dye was used to stain the cells for visualization using a NIKON ECLIPSE TS100 Fluorescence Microscope. Positive panned cultures were expanded for growth and further analysis for homogeneous endosialin expression as described below.

To further determine the ability to isolate EPCs, anti-endosialin-antibody panned HMVEC cells and non-panned HMVEC cultures were prepared for immunostaining using a fluorescent anti-endosialin antibody or fluorescent α-β1-integrin antibody as control. As expected, greater than 90% of the cells stained positive for α-β1-integrin from panned and non-panned cultures (not shown), while greater than 90% of cells stained positive for endosialin from panned cultures while only 5-7% of cells stained positive for endosialin in unpanned HMVEC cultures. These data demonstrate the ability to isolate and enrich viable endosialin expressing cells using endosialin binding proteins such as antibodies.

What is claimed:

1. A method of isolating an endosialin-positive cell comprising contacting a population of cells with an antibody that binds endosialin and isolating cells that bind said antibody, wherein a cell that binds said antibody is an endosialin-positive cell.

2. The method of claim 1 wherein said endosialin-positive cell is isolated from immunosorted cells derived from a tissue.

3. The method of claim 2 wherein said tissue is normal tissue.

4. The method of claim 2 wherein said tissue is malignant tissue.

5. The method of claim 2 wherein said tissue is inflamed tissue.

6. The method of claim 2 wherein said tissue is diseased eye tissue.

7. The method of claim 2 wherein said immunosorted cells are primary culture cells.

8. The method of claim 2 wherein said immunosorted cells are tissue culture cells.

9. The method of claim 1 wherein said endosialin is recombinant endosialin.

10. The method of claim 1 further comprising analyzing an expression profile of said endosialin-positive cell (a) directly after isolation; (b) after culturing said cell; (c) after culturing said cell in a stimulated state with growth factors and/or defined nutrients; or (d) after contacting said cell with a test compound.

11. A hybridoma cell that expresses an antibody that binds endosialin.

12. A cell isolated according to the method of claim 1.

13. The cell of claim 12 wherein said cell is isolated from normal tissue.

14. The cell of claim 12 wherein said cell is isolated from malignant tissue.

15. The cell of claim 12 wherein said cell is isolated from inflamed tissue.

16. The cell of claim 12 wherein said cell is isolated from diseased eye.

17. The cell of claim 12 wherein said cell is isolated from primary cultures.

18. The cell of claim 12 wherein said cell is isolated from tissue culture cells.

19. The cell of claim 12 wherein said cell is a mammalian cell.

20. The cell of claim 12 wherein said cell is a plant cell.

21. The cell of claim 12 wherein said cell is a yeast cell.

22. The cell of claim 12 wherein said cell is a bacterial cell.

23. The cell of claim 12 wherein said cell is a fungal cell.

24. The cell of claim 12 wherein said cell is an insect cell.

25. A method of identifying a compound that stimulates the growth, differentiation, and/or biological activity of an endosialin positive cell of claim 12 comprising contacting said cell with a compound and detecting an increase in the growth, differentiation, or biological activity of the cell in comparison to the growth, differentiation, and/or biological activity of the cell in the absence of the compound.

26. A method of identifying a compound that inhibits the growth, differentiation, and/or biological activity of an endosialin positive cell of claim 12 comprising contacting said cell with a compound and detecting a decrease in the growth, differentiation, or biological activity of the cell in comparison to the growth, differentiation, and/or biological activity of the cell in the absence of the compound.

27. A method of identifying an antibody that specifically binds membrane-bound endosialin comprising contacting a test antibody with an endosialin-positive cell or cell membrane and detecting binding of the antibody to said cell or membrane.


SEQUENCE LISTING (6 sequences)

SEQ ID NO: 1 - DNA, 2660 bp, Mus musculus
gtcaagagcagcggcaggcccgagccgggccagtcggggggcgtcgcgatgctgctgcgcctgctgctggcctgggtggccgcggtgcccgcactgggccaggtcccctggacgccggagcctcgagccgcgtgcggccccagcagctgctacgcgctctttccccggcgccgcacattcctggaagcttggcgggcgtgccgcgaattggggggcaacctggccacaccgcggaccccagaggaggcccagcgtgtggacagcctggtgggggtcgggccggccaacgggctgctatggattgggttgcagcggcaggctaggcaatgccagccgcagcgcccactgcggggcttcatatggaccacgggagaccaggacaccgccttcaccaactgggcccagccggctacggaaggaccctgcccagcccagcgctgtgcagcccttgaggccagcggagagcatcgctggctcgaaggctcgtgcacactggctgtcgatggctacctctgccagtttggttttgagggtgcctgccctgccttgccgcttgaggtgggtcaggccggtcccgctgtctacaccacacccttcaacctggtttccagcgagttcgaatggctgccctttggctccgtggcagctgtgcagtgccaagctggcaggggagcttctctgctgtgcgtgaaacagccttcaggtggcgtgggctggtcccagactggcccgctgtgcccagggactggctgtggtcctgacaatgggggttgcgaacatgagtgtgtggaagaggtggacggtgctgtgtcctgccgctgcagtgaaggcttccgtctagcagcagatgggcacagttgtgaagacccctgtgcccaggccccctgtgagcagcagtgtgaacctggagggccacaaggctatagctgccactgtcgccttggcttccggccagctgaggatgatccacaccgctgcgtggacacggatgagtgccagattgctggtgtgtgccagcagatgtgtgtcaactatgttggtggctttgagtgttactgcagcgagggtcacgagcttgaggcagatggtatcagctgtagccctgcaggagccatgggtgcccaggcttcccaggatctcagagatgagttgctggatgatggagaagaaggggaggatgaagaggagccctgggaggactttgatggcacctggacagaggaacaggggatcctatggctggcacctacacatccacctgactttggcctgccctataggcccaacttcccacaggatggagagcctcagagattgcacctggagcctacctggccacccccacttagtgcccccaggggcccctaccactcctcagtggtgtctgccacacggcccatggtgatctctgccactcgacccacactaccttctgcccacaagacctctgttatttcagctacacgcccacccctgagccctgtccacccacctgccatggcccctgccacacctccagctgtgttctctgagcaccagatccccaaaatcaaggccaattatccagacctgccttttggccacaagcctgggataacctcggccactcacccagcacggtctcctccgtaccagccccccattatctcaaccaactatccccaagtcttccctccccaccaggcccctatgtctccagatacccacactatcacttatttgcctccagtcccccctcaccttgatcctggggataccacttctaaagcccatcaacaccctttgctcccagatgctccaggtatcagaacccaggccccccagctttctgtctcagctctccagccccctcttcctaccaactccaggtcttctgtccatgaaactcctgtgcctgctgccaaccagcccccagccttcccttcttctcccctcccccctcagaggcccactaaccagacctcatctatcagccctacacattcctattccagagcccctctagtcccaagggaaggagttcccagtcccaaatcagtgccacagctgccctcggtgccctccacagcagctccaacagccctggcagagtcaggtcttgcaggccaaagccaaagggatgaccgctggctgctggtggcactcctggtgccaacatgtgtcttcttggtggtgctgcttgccctgggcattgtgtactgcactcgctgtggctcccacgcacccaacaagcggatcacggactgctatcgctgggtcacacatgctgggaacaagagctcaacagaacccatgccccccagaggcagccttacaggggtacagacctgtagaaccagtgtgtgatggggtgcagatgcccctttgtgggatagaagaaaaggacttgctttggacacatggctgagaccacaccaaggacttatgggggctgcccagctgacagaggaggttctgttctttgagcccagcatccatggcaaaggacacaccaggactccaggacctcaaggggtgggtgctgggatcttctccaataaatggggtgccaacctcacccaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 765 aa, Mus musculus
MLLRLLLAWVAAVPALGQVPWTPEPRAACGPSSCYALFPRRRTFLEAWRACRELGGNLATPRTPEEAQRVDSLVGVGPANGLLWIGLQRQARQCQPQRPLRGFIWTTGDQDTAFTNWAQPATEGPCPAQRCAALEASGEHRWLEGSCTLAVDGYLCQFGFEGACPALPLEVGQAGPAVYTTPFNLVSSEFEWLPFGSVAAVQCQAGRGASLLCVKQPSGGVGWSQTGPLCPGTGCGPDNGGCEHECVEEVDGAVSCRCSEGFRLAADGHSCEDPCAQAPCEQQCEPGGPQGYSCHCRLGFRPAEDDPHRCVDTDECQIAGVCQQMCVNYVGGFECYCSEGHELEADGISCSPAGAMGAQASQDLRDELLDDGEEGEDEEEPWEDFDGTWTEEQGILWLAPTHPPDFGLPYRPNFPQDGEPQRLHLEPTWPPPLSAPRGPYHSSVVSATRPMVISATRPTLPSAHKTSVISATRPPLSPVHPPAMAPATPPAVFSEHQIPKIKANYPDLPFGHKPGITSATHPARSPPYQPPIISTNYPQVFPPHQAPMSPDTHTITYLPPVPPHLDPGDTTSKAHQHPLLPDAPGIRTQAPQLSVSALQPPLPTNSRSSVHETPVPAANQPPAFPSSPLPPQRPTNQTSSISPTHSYSRAPLVPREGVPSPKSVPQLPSVPSTAAPTALAESGLAGQSQRDDRWLLVALLVPTCVFLVVLLALGIVYCTRCGSHAPNKRITDCYRWVTHAGNKSSTEPMPPRGSLTGVQTCRTSV

SEQ ID NO: 3 - DNA, 2576 bp, Homo sapiens
agtccgggggcatcgcgatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtgatggggtgcagacccccctcatggagtatggggcgctggacacatggccggggctgcaccagggacccatgggggctgcccagctggacagatggcttcctgctccccaggcccagccagggtcctctctcaaccactagacttggctctcaggaactctgcttcctggcccagcgctcgtgaccaaggatacaccaaagcccttaagacctcagggggcgggtgctggggtcttctccaataaatggggtgtcaaccttacccaaggaaaaaaaaaaaaaaaaaa

SEQ ID NO: 4 - protein, 757 aa, Homo sapiens
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 5 - DNA, 2397 bp, Rattus norvegicus
atgctgctgcgcctgctgctggcctgggcggccgcggtgcccgcactgggccaggccccctggacgccggagcctagagccgcctgcggccccagcagctgctacgctctctttccccggcgccgcacattcctggaggcttggcggtcgtgccgcgaattggggggcaacctggccacaccgaggaccccggaggaggcccgacgtgtggacagcctggtgggcgtcggacccgccaacgggctgctatggattgggttgcagcggcaggctcggcaatgccagccacagcgcccactgcggggcttcatatggaccacgggagaccaggacaccgccttcactaactgggcccagccggctacggaaggaccctgcccggcccagcgctgtgctgcccttgaggccagcggagaacatcgctggctcgaaggctcgtgcacactggctgtcgatggctacctctgccagtttggttttgagggtgcctgtcctgccttgccgcttgaggtgggccaagccggtccagctatctacaccacacccttcaacctggtttccagtgagttcgaatggctaccctttggctccgtggcagctgtgcagtgccaagctggcaggggaacgtctctgttgtgtgtgaaacaaccttcaggtggcgttggctggtcccagactggcccactgtgtccagggactggctgtggtcctgacaatgggggttgcgaacatgaatgtgtggaagagttggatggcggtatgtcctgccgctgcagtgaaggcttccgtctagcagcagatgggcacagttgtgaagacccttgtgcccaggccccctgtgagcagcagtgtgagcctggtgggccacaaggctacagctgccactgtcgcctaggcttccggccagctgaggatgagccacaccgctgcgtggacacggatgagtgccagattgctggtgtgtgccagcagatgtgtgtcaactatgttggtggctttgagtgttactgcagggagggtcatgagcttgaggcagatggtatcagttgtagccctgcaggagctatgggtgcccaggcttcccaggatcttagagacgagttgctggatgatggagaagaaggggaggatgaagaggagccctgggaggacttcgatggcacctggacagaggagcaggggaccctatggatggcacctacacatccgcctgactttggcctgccctataggcccaacttcccacaggatggagagcctcagagattgcacctggagcctacctggccacccccacttagcgcccccaggggcccctaccactcctcagtggtgtctgccacacggcccatggtaatctctgccactcgacccacacaaccttctgcccgaaagacctctgttatttcagccacacacctaccccttaaccctgtccacccacctgccctagcccctaccacacctccagccgtgctccctgagcaccagatccccaaaatcaaggccagttatccagacttgccttttggccacaagcctgggataacctcagccactcacccagcacagcctcctcctcaccagccccccatcatctcaacgaaatatccccaagtcttccctccccagcaggcccctatgtctccagacacccacactatcactaatttgcctctaatcccatctcaccttgaccctggggataccacttcccaagccggtcaccatcctttgctcccagatgttccaggtatcagaacccaggctccccaggtttctgtctcagctctccagccctctctgcctaccaactccaggtcttctgtccatgaaccccctgtgcctactgccaaccagcccccagccttcccttctcccctgccccctcagagccccattaaccagacctcatctatcagccctacacactcctattccagagcccctcaggtcccaagggaaggagctcccagtcccaaatcagtgccaaggctgcactcagtggcccccacagcagctccaacagccctggcagagttgggtcttgcaggccaaagccagagagatgaccgatggctgctggtggcactcttggtaccaacgtgtgtcttcttggtggtcctgctcgcattgggcattgtgtactgcactcgctgtggctcccatacgcccaacaagcgtatcactgactgctatcgctgggtcacgcatgctgggaacaagagctcaacagaacccatgccccccagatggacagagaaggttctgttccttgaacccagcattcatggcaaaggacacactgaaggactccaggacctcaaggggtgggtgctgggatcttctccaataaatggcgtgccaacctcacccaaagtccgtgatccccgctga

SEQ ID NO: 6 - protein, 798 aa, Rattus norvegicus
MLLRLLLAWAAAVPALGQAPWTPEPRAACGPSSCYALFPRRRTFLEAWRSCRELGGNLATPRTPEEARRVDSLVGVGPANGLLWIGLQRQARQCQPQRPLRGFIWTTGDQDTAFTNWAQPATEGPCPAQRCAALEASGEHRWLEGSCTLAVDGYLCQFGFEGACPALPLEVGQAGPAIYTTPFNLVSSEFEWLPFGSVAAVQCQAGRGTSLLCVKQPSGGVGWSQTGPLCPGTGCGPDNGGCEHECVEELDGGMSCRCSEGFRLAADGHSCEDPCAQAPCEQQCEPGGPQGYSCHCRLGFRPAEDEPHRCVDTDECQIAGVCQQMCVNYVGGFECYCREGHELEADGISCSPAGAMGAQASQDLRDELLDDGEEGEDEEEPWEDFDGTWTEEQGTLWMAPTHPPDFGLPYRPNFPQDGEPQRLHLEPTWPPPLSAPRGPYHSSVVSATRPMVISATRPTQPSARKTSVISATHLPLNPVHPPALAPTTPPAVLPEHQIPKIKASYPDLPFGHKPGITSATHPAQPPPHQPPIISTKYPQVFPPQQAPMSPDTHTITNLPLIPSHLDPGDTTSQAGHHPLLPDVPGIRTQAPQVSVSALQPSLPTNSRSSVHEPPVPTANQPPAFPSPLPPQSPINQTSSISPTHSYSRAPQVPREGAPSPKSVPRLHSVAPTAAPTALAELGLAGQSQRDDRWLLVALLVPTCVFLVVLLALGIVYCTRCGSHTPNKRITDCYRWVTHAGNKSSTEPMPPRWTEKVLFLEPSIHGKGHTEGLQDLKGWVLGSSPINGVPTSPKVRDPR